SEQUENCE LISTING (53 sequences)

SEQ ID NO: 1 - DNA, 5317 bp, Artificial Sequence
  Synthetic sequence
  promoter (214)..(232): Bacteriophage T7 gene 1 promoter
  misc_feature (365)..(372): AsiSI restriction endonuclease site
  misc_feature (481)..(488): NotI restriction endonuclease site
  terminator (562)..(609): Bacteriophage T7 transcription terminator
  promoter (935)..(953): Bacteriophage T7 gene 1 promoter
  gene (1026)..(1415): Bacteriophage MS2 coat protein
  terminator (1448)..(1495): Bacteriophage T7 transcription terminator
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcacgcggacaactactacagggtttaaacctttcggattataacatcacatctaggcgcgcctgacgatcaaccataccagacggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 2 - DNA, 691 bp, Solenopsis invicta
gccatctgcaattatcaacgcctttcttaacgtcaacgacagacatcttacgctatgatccagtccaccacagttcggaaaaaatagtgacgatcaataagatatttttcgaattagcatttgattatattggcaataacttgtacacaacaaatactgtaaaccaatctatcgaggtaatcaacttaaacaccaaggcaatgacggccttttattttaaggatgaggttcctaaatatattgcactagctcctgaagaaagcaagatgttcgtagcctttcaaaaatcaatgcattcgatcagtggtttgactttatatgagatgcaaatgaacggactcggtaaaagaaagctaattagggaaggattaattggtccacaattaccaatgtactatgacagagatagtaaaacactttttgtgagtgatttgctcccaggttacatttactcgcattcggcccaagacacgcgtatacttcgttctggactaaagagcccccacagtcttactgtcgccggcgacaatcttttctggatcgaatcacagaataaattatactcgacaaattttagaacagcctctgtaaaacagaagactgttgagtttgatctctctaaattaaacgataatatgacatctttacctggtcatttgacaccttattcacgcgatgcacagtatgtggt

SEQ ID NO: 3 - DNA, 345 bp, Solenopsis invicta
ccatctgcaattatcaacgcctttcttaacgtcaacgacagacatcttacgctatgatccagtccaccacagttcggaaaaaatagtgacgatcaataagatatttttcgaattagcatttgattatattggcaataacttgtacacaacaaatactgtaaaccaatctatcgaggtaatcaacttaaacaccaaggcaatgacggccttttattttaaggatgaggttcctaaatatattgcactagctcctgaagaaagcaagatgttcgtagcctttcaaaaatcaatgcattcgatcagtggtttgactttatatgagatgcaaatgaacggactcggtaa

SEQ ID NO: 4 - DNA, 344 bp, Solenopsis invicta
agaaagctaattagggaaggattaattggtccacaattaccaatgtactatgacagagatagtaaaacactttttgtgagtgatttgctcccaggttacatttactcgcattcggcccaagacacgcgtatacttcgttctggactaaagagcccccacagtcttactgtcgccggcgacaatcttttctggatcgaatcacagaataaattatactcgacaaattttagaacagcctctgtaaaacagaagactgttgagtttgatctctctaaattaaacgataatatgacatctttacctggtcatttgacaccttattcacgcgatgcacagtatgtggt

SEQ ID NO: 5 - DNA, 1548 bp, Artificial Sequence
  Synthetic DNA construct
  misc_feature (1)..(8): AsiSI restriction endonuclease site
  misc_feature (9)..(699): SEQ ID NO. 2 sense sequence
  misc_feature (700)..(849): non-homologous loop sequence
  misc_feature (850)..(1540): SEQ ID NO. 2 anti-sense sequence
  misc_feature (1541)..(1548): NotI restriction endonuclease site
gcgatcgcgccatctgcaattatcaacgcctttcttaacgtcaacgacagacatcttacgctatgatccagtccaccacagttcggaaaaaatagtgacgatcaataagatatttttcgaattagcatttgattatattggcaataacttgtacacaacaaatactgtaaaccaatctatcgaggtaatcaacttaaacaccaaggcaatgacggccttttattttaaggatgaggttcctaaatatattgcactagctcctgaagaaagcaagatgttcgtagcctttcaaaaatcaatgcattcgatcagtggtttgactttatatgagatgcaaatgaacggactcggtaaaagaaagctaattagggaaggattaattggtccacaattaccaatgtactatgacagagatagtaaaacactttttgtgagtgatttgctcccaggttacatttactcgcattcggcccaagacacgcgtatacttcgttctggactaaagagcccccacagtcttactgtcgccggcgacaatcttttctggatcgaatcacagaataaattatactcgacaaattttagaacagcctctgtaaaacagaagactgttgagtttgatctctctaaattaaacgataatatgacatctttacctggtcatttgacaccttattcacgcgatgcacagtatgtggtcctctagctgctttacaaagtactggttccctttccagcgggatgctttatctaaacgcaatgagagaggtattcctcaggccacatcgcttcctagttccgctgggatccatcgttggcggccgaagccgccattccatagtgagttctaccacatactgtgcatcgcgtgaataaggtgtcaaatgaccaggtaaagatgtcatattatcgtttaatttagagagatcaaactcaacagtcttctgttttacagaggctgttctaaaatttgtcgagtataatttattctgtgattcgatccagaaaagattgtcgccggcgacagtaagactgtgggggctctttagtccagaacgaagtatacgcgtgtcttgggccgaatgcgagtaaatgtaacctgggagcaaatcactcacaaaaagtgttttactatctctgtcatagtacattggtaattgtggaccaattaatccttccctaattagctttcttttaccgagtccgttcatttgcatctcatataaagtcaaaccactgatcgaatgcattgatttttgaaaggctacgaacatcttgctttcttcaggagctagtgcaatatatttaggaacctcatccttaaaataaaaggccgtcattgccttggtgtttaagttgattacctcgatagattggtttacagtatttgttgtgtacaagttattgccaatataatcaaatgctaattcgaaaaatatcttattgatcgtcactattttttccgaactgtggtggactggatcatagcgtaagatgtctgtcgttgacgttaagaaaggcgttgataattgcagatggcgcggccgc

SEQ ID NO: 6 - DNA, 6088 bp, Artificial Sequence
  Expression plasmid
  promoter (38)..(56): Bacteriophage T7 gene 1promoter
  promoter (38)..(56): Bacteriophage T7 gene 1 promoter
  misc_feature (60)..(67): AsiI restriction endonuclease site
  misc_feature (64)..(1601): AsiSI-NotI synthetic DNA fragment
  misc_feature (1599)..(1607): NotI restriction endonuclease site
  terminator (1608)..(1655): Bacteriophage T7 transcription terminator
  promoter (1706)..(1724): Bacteriophage T7 gene 1 promoter
  gene (1797)..(2186): Bacteriophage MS2 coat protein
  terminator (2219)..(2266): Bacteriophage T7 transcription terminator
ttcttagctccggcaagcaattaagaacttccgaaattaatacgactcactatagggaggcgatcgcgccatctgcaattatcaacgcctttcttaacgtcaacgacagacatcttacgctatgatccagtccaccacagttcggaaaaaatagtgacgatcaataagatatttttcgaattagcatttgattatattggcaataacttgtacacaacaaatactgtaaaccaatctatcgaggtaatcaacttaaacaccaaggcaatgacggccttttattttaaggatgaggttcctaaatatattgcactagctcctgaagaaagcaagatgttcgtagcctttcaaaaatcaatgcattcgatcagtggtttgactttatatgagatgcaaatgaacggactcggtaaaagaaagctaattagggaaggattaattggtccacaattaccaatgtactatgacagagatagtaaaacactttttgtgagtgatttgctcccaggttacatttactcgcattcggcccaagacacgcgtatacttcgttctggactaaagagcccccacagtcttactgtcgccggcgacaatcttttctggatcgaatcacagaataaattatactcgacaaattttagaacagcctctgtaaaacagaagactgttgagtttgatctctctaaattaaacgataatatgacatctttacctggtcatttgacaccttattcacgcgatgcacagtatgtggtcctctagctgctttacaaagtactggttccctttccagcgggatgctttatctaaacgcaatgagagaggtattcctcaggccacatcgcttcctagttccgctgggatccatcgttggcggccgaagccgccattccatagtgagttctaccacatactgtgcatcgcgtgaataaggtgtcaaatgaccaggtaaagatgtcatattatcgtttaatttagagagatcaaactcaacagtcttctgttttacagaggctgttctaaaatttgtcgagtataatttattctgtgattcgatccagaaaagattgtcgccggcgacagtaagactgtgggggctctttagtccagaacgaagtatacgcgtgtcttgggccgaatgcgagtaaatgtaacctgggagcaaatcactcacaaaaagtgttttactatctctgtcatagtacattggtaattgtggaccaattaatccttccctaattagctttcttttaccgagtccgttcatttgcatctcatataaagtcaaaccactgatcgaatgcattgatttttgaaaggctacgaacatcttgctttcttcaggagctagtgcaatatatttaggaacctcatccttaaaataaaaggccgtcattgccttggtgtttaagttgattacctcgatagattggtttacagtatttgttgtgtacaagttattgccaatataatcaaatgctaattcgaaaaatatcttattgatcgtcactattttttccgaactgtggtggactggatcatagcgtaagatgtctgtcgttgacgttaagaaaggcgttgataattgcagatggcgcggccgcctagcataaccccttggggcctctaaacgggtcttgaggggttttttgagaaacggccgaatacacctgttcggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 7 - DNA, 858 bp, Artificial Sequence
  Synthetic DNA construct
  misc_feature (1)..(8): AsiSI restriction endonuclease site
  misc_feature (10)..(354): SEQ ID NO. 3 sense sequence
  misc_feature (355)..(504): non-homologous loop sequence
  misc_feature (505)..(849): SEQ ID NO. 3 anti-sense sequence
  misc_feature (849)..(858): NotI restriction endonuclease site
gcgatcgcgccatctgcaattatcaacgcctttcttaacgtcaacgacagacatcttacgctatgatccagtccaccacagttcggaaaaaatagtgacgatcaataagatatttttcgaattagcatttgattatattggcaataacttgtacacaacaaatactgtaaaccaatctatcgaggtaatcaacttaaacaccaaggcaatgacggccttttattttaaggatgaggttcctaaatatattgcactagctcctgaagaaagcaagatgttcgtagcctttcaaaaatcaatgcattcgatcagtggtttgactttatatgagatgcaaatgaacggactcggtaacctctagctgctttacaaagtactggttccctttccagcgggatgctttatctaaacgcaatgagagaggtattcctcaggccacatcgcttcctagttccgctgggatccatcgttggcggccgaagccgccattccatagtgagttctttaccgagtccgttcatttgcatctcatataaagtcaaaccactgatcgaatgcattgatttttgaaaggctacgaacatcttgctttcttcaggagctagtgcaatatatttaggaacctcatccttaaaataaaaggccgtcattgccttggtgtttaagttgattacctcgatagattggtttacagtatttgttgtgtacaagttattgccaatataatcaaatgctaattcgaaaaatatcttattgatcgtcactattttttccgaactgtggtggactggatcatagcgtaagatgtctgtcgttgacgttaagaaaggcgttgataattgcagatggcgcggccgc

SEQ ID NO: 8 - DNA, 5398 bp, Artificial Sequence
  Expression plasmid
  promoter (38)..(56): Bacteriophage T7 gene 1 promoter
  misc_feature (60)..(67): AsiSI restriction endonuclease site
  misc_feature (68)..(909): AsiSI-NotI synthetic DNA fragment
  misc_feature (908)..(917): NotI restriction endonuclease site
  terminator (918)..(965): Bacteriophage T7 transcription terminator
  promoter (1016)..(1034): Bacteriophage gene 1 promoter
  gene (1107)..(1496): Bacteriophage MS2 coat protein gene
  terminator (1529)..(1576): Bacteriophage T7 transcription terminator
ttcttagctccggcaagcaattaagaacttccgaaattaatacgactcactatagggaggcgatcgcgccatctgcaattatcaacgcctttcttaacgtcaacgacagacatcttacgctatgatccagtccaccacagttcggaaaaaatagtgacgatcaataagatatttttcgaattagcatttgattatattggcaataacttgtacacaacaaatactgtaaaccaatctatcgaggtaatcaacttaaacaccaaggcaatgacggccttttattttaaggatgaggttcctaaatatattgcactagctcctgaagaaagcaagatgttcgtagcctttcaaaaatcaatgcattcgatcagtggtttgactttatatgagatgcaaatgaacggactcggtaacctctagctgctttacaaagtactggttccctttccagcgggatgctttatctaaacgcaatgagagaggtattcctcaggccacatcgcttcctagttccgctgggatccatcgttggcggccgaagccgccattccatagtgagttctttaccgagtccgttcatttgcatctcatataaagtcaaaccactgatcgaatgcattgatttttgaaaggctacgaacatcttgctttcttcaggagctagtgcaatatatttaggaacctcatccttaaaataaaaggccgtcattgccttggtgtttaagttgattacctcgatagattggtttacagtatttgttgtgtacaagttattgccaatataatcaaatgctaattcgaaaaatatcttattgatcgtcactattttttccgaactgtggtggactggatcatagcgtaagatgtctgtcgttgacgttaagaaaggcgttgataattgcagatggcgcggccgcctagcataaccccttggggcctctaaacgggtcttgaggggttttttgagaaacggccgaatacacctgttcggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 9 - DNA, 856 bp, Artificial Sequence
  Synthetic DNA construct
  misc_feature (1)..(8): AsiSI restriction endonuclease site
  misc_feature (10)..(353): SEQ ID NO. 4 sense sequence
  misc_feature (354)..(503): non-homologous loop sequence
  misc_feature (504)..(847): SEQ ID NO. 4 anti-sense sequence
  misc_feature (849)..(856): NotI restriction endonuclease site
gcgatcgcaagaaagctaattagggaaggattaattggtccacaattaccaatgtactatgacagagatagtaaaacactttttgtgagtgatttgctcccaggttacatttactcgcattcggcccaagacacgcgtatacttcgttctggactaaagagcccccacagtcttactgtcgccggcgacaatcttttctggatcgaatcacagaataaattatactcgacaaattttagaacagcctctgtaaaacagaagactgttgagtttgatctctctaaattaaacgataatatgacatctttacctggtcatttgacaccttattcacgcgatgcacagtatgtggtcctctagctgctttacaaagtactggttccctttccagcgggatgctttatctaaacgcaatgagagaggtattcctcaggccacatcgcttcctagttccgctgggatccatcgttggcggccgaagccgccattccatagtgagttctaccacatactgtgcatcgcgtgaataaggtgtcaaatgaccaggtaaagatgtcatattatcgtttaatttagagagatcaaactcaacagtcttctgttttacagaggctgttctaaaatttgtcgagtataatttattctgtgattcgatccagaaaagattgtcgccggcgacagtaagactgtgggggctctttagtccagaacgaagtatacgcgtgtcttgggccgaatgcgagtaaatgtaacctgggagcaaatcactcacaaaaagtgttttactatctctgtcatagtacattggtaattgtggaccaattaatccttccctaattagctttcttgcggccgc

SEQ ID NO: 10 - DNA, 5396 bp, Artificial Sequence
  Expression plasmid
  promoter (38)..(56): Bacteriophage T7 gene 1 promoter
  misc_feature (60)..(67): AsiSI restriction endonuclease site
  misc_feature (68)..(907): AsiSI-NotI synthetic DNA fragment
  misc_feature (908)..(914): NotI restriciton endonuclease site
  terminator (916)..(963): Bacteriophage T7 transcription terminator
  promoter (1014)..(1032): Bacteriophage T7 gene 1 promoter
  gene (1105)..(1494): Bacteriophage T7 MS2 coat protein gene
  terminator (1527)..(1574): Bacteriophage T7 transcription terminator
ttcttagctccggcaagcaattaagaacttccgaaattaatacgactcactatagggaggcgatcgcaagaaagctaattagggaaggattaattggtccacaattaccaatgtactatgacagagatagtaaaacactttttgtgagtgatttgctcccaggttacatttactcgcattcggcccaagacacgcgtatacttcgttctggactaaagagcccccacagtcttactgtcgccggcgacaatcttttctggatcgaatcacagaataaattatactcgacaaattttagaacagcctctgtaaaacagaagactgttgagtttgatctctctaaattaaacgataatatgacatctttacctggtcatttgacaccttattcacgcgatgcacagtatgtggtcctctagctgctttacaaagtactggttccctttccagcgggatgctttatctaaacgcaatgagagaggtattcctcaggccacatcgcttcctagttccgctgggatccatcgttggcggccgaagccgccattccatagtgagttctaccacatactgtgcatcgcgtgaataaggtgtcaaatgaccaggtaaagatgtcatattatcgtttaatttagagagatcaaactcaacagtcttctgttttacagaggctgttctaaaatttgtcgagtataatttattctgtgattcgatccagaaaagattgtcgccggcgacagtaagactgtgggggctctttagtccagaacgaagtatacgcgtgtcttgggccgaatgcgagtaaatgtaacctgggagcaaatcactcacaaaaagtgttttactatctctgtcatagtacattggtaattgtggaccaattaatccttccctaattagctttcttgcggccgcctagcataaccccttggggcctctaaacgggtcttgaggggttttttgagaaacggccgaatacacctgttcggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 11 - DNA, 226 bp, Solenopsis invicta
tatgaaatgggtacatgtgaaagggaatacatataaatcggaaaagcttgagttatttcataaattacaggaaaaaaagtaaaaagagaaaaacatgcaacaacacacaatattaaaaattaaagtaaaagcaaacatgacatcttttattccatctacaagatataattgtaaagtgagattgtttgaatctagaagtcgaataaggcaaacgagtcgtactacg

SEQ ID NO: 12 - DNA, 105 bp, Solenopsis invicta
tagaaagcaggaatactgtacaagatatatgccgtaaaattttggatagagatattggtcttgtcaaaccttatagaagcattaacttagatcatgttattcctg

SEQ ID NO: 13 - DNA, 147 bp, Solenopsis invicta
ttttgataaattgaaacatacaatagaaaatactagatgtaaaaaaactcaagagaacaagaaaccaaagtataaacatcaaattgatgtctgtttagtgcaagctttctttagtttgttattgtataaaattgtccctcttgaatt

SEQ ID NO: 14 - DNA, 207 bp, Solenopsis invicta
atttggcacatcaagaaaccgaaagcttataaaatccacaatatatcgtcttttaaagacaaagccaaacaagataccagtatcacagccttttaaaagaaaaaaatcaaaaagaacagatgccattgggggcatactagatttggaacatgctaaaccatttttattaaaaaagctggatatttctcaaattcagtggttacattc

SEQ ID NO: 15 - DNA, 114 bp, Solenopsis invicta
atatgcaagaagaatttataaaagaaaagaaggatactaataatcttgtgccctatgcaaagttaaataacacatatattgggatatataaatttcttccaagttcttctggtt

SEQ ID NO: 16 - DNA, 108 bp, Solenopsis invicta
acggcggatcgagtgagagtagatctccgagcaacgattttggttcctgtatcgacggcaaatgtatcaagcgcacctcgcaggatatcgccagcggcatgtggttcg

SEQ ID NO: 17 - DNA, 224 bp, Solenopsis invicta
gaacatcacctggacccttcagaatagggatccctggagactcctgaaagacggcaatctacaggaatctcgttccacggtcgacggtgtcgttcacgcaaatccgcaatatgagaattatacgaacatgctcagccccttgcttagagaagagagcgccgttgatcgccgggactccctatacatcgtgctgccgatcacggtgatttacgtcgtgatcttct

SEQ ID NO: 18 - DNA, 141 bp, Solenopsis invicta
cgaaactctcccgggcggtgaagttcgtgatagcgatttggctgctggccctctgcttcgccgtgccacaggccatccaattcggcatcatctactcccactacaacggcactgtcatcttggatagcgccaggtgctcgc

SEQ ID NO: 19 - DNA, 109 bp, Solenopsis invicta
tcgttaagcaaacaaggtttaaacaacgaaggcaacaacaacgcaaatgaatgtctactgagaattcacaggtcaccgaaggctgtcactttaggaatcttcacggaga

SEQ ID NO: 20 - DNA, 106 bp, Solenopsis invicta
agatttcatcgaacaatatgacgaaaatgacgaagccaaaaccggaaaacgtaattgcgatgccaccacctcttgagagtcatccgagcatcgagagcgccaatac

SEQ ID NO: 21 - DNA, 141 bp, Solenopsis invicta
acattcttggcacaccatgcaaaatcattagcgttaatgattctcatgatgaaaataaatggttttatccaagaggaaaaggttcctgttctcaagttgacctgcatcaatttagtttagacattcatcagcaagcttatc

SEQ ID NO: 22 - DNA, 177 bp, Solenopsis invicta
cattattatgcgcatcattaactgtcattggttttatacttggacaagttgcggaaggtcagtggaaatgggatgaagatttacaattggaaatgacttctgcctttttcaccggagtatatggaatgtggaatatttacattattgcattattatgcctctatgcaccttctcaca

SEQ ID NO: 23 - DNA, 137 bp, Solenopsis invicta
ggttgggtgggtaaaacatgtagccaacgtgcttgccagtatggtttgtacggtcccaactgtacgaaggtttgcgagtgcgaaaacgataatacagagctctgccatccctggaccgggcaatgttcttgcaaaat

SEQ ID NO: 24 - DNA, 103 bp, Solenopsis invicta
tctatgacagtagagttgtcggcaagtattgcgagaaacgcgatccccatttggcttgcatcgcgtacgagcgtggtcagtgtgaccgcgagctgataagcgt

SEQ ID NO: 25 - DNA, 71 bp, Solenopsis invicta
gttgtatgccaaaaaagtctcgtatacgcccgactatatattcctcttgcgaaatgttatgaggattaatc

SEQ ID NO: 26 - DNA, 101 bp, Solenopsis invicta
tgctccacgcatggatcatactagatcagtggcgtatttcacgcgaactaaccatctgcaactagtgaaaccgtatttgagatcggtccaggctctcaaca

SEQ ID NO: 27 - DNA, 71 bp, Camponotus floridanus
cctctacgccaaaaaagtctcgtatacacccgactatatatttctcttgcgaaatgtcatgaggattaatc

SEQ ID NO: 28 - DNA, 101 bp, Camponotus floridanus
cgctccacgtatggatcatactagatcagtggcgtactttacgagaaccggccatttacaactagtaaaaccatacttgagatcggttcaagctctcaaca

SEQ ID NO: 29 - DNA, 71 bp, Linepithema humile
tctctacgccaagaaagtctcgtatactcccgactatatatttctcttgcggaatgtcatgaggattaatc

SEQ ID NO: 30 - DNA, 101 bp, Linepithema humile
tgctccacgtatggatcatactagatcagtggcatactttacgagaaccggccatctacaactagtgaaaccatacttgagatcggttcaagctctcaata

SEQ ID NO: 31 - DNA, 71 bp, Monomorium pharaonis
gttatacgccaagaaagtctcgtatacacccgactatatattcctcttgcggaacgtcatgaggattaatc

SEQ ID NO: 32 - DNA, 101 bp, Monomorium pharaonis
tgctccgcgcatggatcatactagatcagtggcgtatttcacgcgaactggtcatctgcaattagtgaaaccatatttgagatcagttcaggctctcaata

SEQ ID NO: 33 - DNA, 127 bp, Solenopsis invicta
acggagcaggatggagtctaccgacatgtatcaagatgagagcgataacgatgcaaaagactctgcaaatctcatgatgtcgatcccaatagtgaacgaactatttgatgtgcattgcaaagttgga

SEQ ID NO: 34 - DNA, 101 bp, Solenopsis invicta
gcaagaatatcacgtggagaaattacgagaaagtacaagagaagtaaacgaggagagcactgaagtacatggtgtaaagcggaaaagatctaatgaaaata

SEQ ID NO: 35 - DNA, 107 bp, Solenopsis invicta
tgaaaataacatgagcctccaacctgattttcttgctaagaaaaatgtaaatagaaaatgtcattgttttgggaaacctaaaatatgcacgatatgcttaaaagaac

SEQ ID NO: 36 - DNA, 119 bp, Solenopsis invicta
agcaagcatagcgttcaaaagaagattgatagattagttgaagagaaccgaatgttaacaatgaggttggaaagttctaacgtaagtcctgagattattgctgaattacaagaaaaaga

SEQ ID NO: 37 - DNA, 105 bp, Solenopsis invicta
atgaaactgaaaaggaaaaattatccgctcgcattaaagaattagatgaatcttatgggcaaaataaaacacaactggatgtagttacgatagagagaaatcaat

SEQ ID NO: 38 - DNA, 153 bp, Solenopsis invicta
gtagtgtcattcaatttcagacaattttcattcgaaacactttggagagccaaacgcaatgatggatctgttgacagaaaatggaaaaggacgtttacccgattacgaggtgcaaggagcgaaaaaagtgagcttcagtccgtattctgacaa

SEQ ID NO: 39 - DNA, 137 bp, Solenopsis invicta
agttatttgaatattccaacgttttgttatcaaattctctcttgaacttcctttgaaggaaagaatgcactatgtggaataaggtagcttgatttttttttacagattttaaataatgtataaaagacaaaagtctg

SEQ ID NO: 40 - DNA, 220 bp, Solenopsis invicta
aggaaagtcatggtgcatctacatttgatgttgccattgctatttttaagcagccatgcacgaacgaggactttttaacggaagcgttgagaattctgaaacccgatggttcgcttgttatttatgaaccactgtcagcagacaaaaagccagatacagtacttacatatcccgaaaggatatccagactaaagttgtctggctttaaagtaaaagatac

SEQ ID NO: 41 - DNA, 110 bp, Solenopsis invicta
actcagctaactgtcgattcttgaatttgactttcctttatcaaaatacagttacaatgaatgcgtattatttttatttgtgtaagtaactgtcctgttatgtgattcta

SEQ ID NO: 42 - DNA, 300 bp, Solenopsis invicta
ctaaaagagagcaaacatgtgcgacgaggaagttgccgcgctcgtagttgacaatggatccggcatgtgcaaggctggcttcgccggggacgacgcaccccgcgccgtcttcccctcgatcgtcggcaggccacgccaccaaggtgtcatggtcggtatgggacagaaggattcttacgtcggtgacgaggctcaatcgaaaaggggtattctaaccttgaaatatccgatcgagcacggtatcgtcaccaactgggacgacatggagaagatctggcatcacacgttctacaacgaact

SEQ ID NO: 43 - DNA, 300 bp, Solenopsis invicta
cgatctctctccctcgactctaacaccagcgaaagtaacagccaatcaagatgtgtgacgatgatgttgcggcattagtcgtggacaatgggtccggtatgtgcaaggctggattcgcgggggatgatgcaccacgcgctgtgtttcccagcatcgtcggtcgtcctcgtcatcagggtgtgatggtcggtatgggtcaaaaagacagttatgttggcgacgaggcgcaaagtaagagaggtatattgacactaaagtatcctatagaacatggcattattactaattgggatgacatgg

SEQ ID NO: 44 - DNA, 387 bp, Solenopsis invicta
accgaaactctctttcgagagaatcagaaagattacgacagaccgcgacagactggagtggacgaaggcagtggacacggcagaattgggcagaatatatcaatatatacacggttgataacggacgagcgttcggctggtgcggctggatcagggcccaggggtacaacggggctcgggaaacggaatacaccaatacacgtcggagctgcgcgggcacaagcgagagtgtcgggtcagtcgggtgtcgcgcgcgcgtatcacgtggtatcactggtatcacggatcatcacggatcatcacggatcgtcacgggttattatagacggtggacggttgtaaaacgcacggagagaatatgtcgcgcagagcgatcaaagcgatcaa

SEQ ID NO: 45 - DNA, 238 bp, Solenopsis invicta
cgagctcggagacttcctcgcaggtcatcgcacgcacgcacgcacgcacgcacaagcgtcgcgatcggttcccgactctcccccggccaatcgataccgtctcgctctttcgccgcgcccgagatcaaacggcgcgatcttgaacaccgaccaacacgcgggtttcggcatggctccggcaatcaagatcctcctcgtcctgctctgtaccctgctcgccgtggcgatggccaccgag

SEQ ID NO: 46 - DNA, 73 bp, Solenopsis invicta
cgagcgtgctcaacagctttcgatacgcgtaactaatgccagtgcacgtttgcgttcggaagagaatgagtaa

SEQ ID NO: 47 - DNA, 81 bp, Solenopsis invicta
gaagagcattgtcgagcgtgctcaacagctttcgatacgcgtaactaatgccagtgcacgtttgcgttcggaagagaatga

SEQ ID NO: 48 - DNA, 73 bp, Camponotus floridanus
cgaacgtgctcaacagctttcgatacgtgtgacaaatgccagtgcacgtttgcgatcggaagagaacgagtaa

SEQ ID NO: 49 - DNA, 81 bp, Camponotus floridanus
aaaagctatcgtcgaacgtgctcaacagctttcgatacgtgtgacaaatgccagtgcacgtttgcgatcggaagagaacga

SEQ ID NO: 50 - DNA, 73 bp, Linepithema humile
tgagcgtgcacaacagctctcgatacgtgtgactaatgccagtgcacgtttacgttcggaagagaacgagtaa

SEQ ID NO: 51 - DNA, 81 bp, Linepithema humile
gaaagctatcgttgagcgtgcacaacagctctcgatacgtgtgactaatgccagtgcacgtttacgttcggaagagaacga

SEQ ID NO: 52 - DNA, 73 bp, Monomorium pharaonis
cgagcgtgctcaacaactttcgatacgcgtgacaaatgccagtgcacgtttacgttcggaagagaacgagtaa

SEQ ID NO: 53 - DNA, 81 bp, Monomorium pharaonis
gaagagcattgtcgagcgtgctcaacaactttcgatacgcgtgacaaatgccagtgcacgtttacgttcggaagagaacga

(19) United States
(12) Patent Application Publication
(10) Pub. No.: US 2019/0029265 A1
(43) Pub. Date: Jan. 31, 2019

(54) Methods and Compositions for Controlling Ants

(71) Applicant: APSE, INC., St. Louis, MO (US)

(72) Inventors: John L. KILLMER, St. Louis, MO (US); Anil KUMAR, St. Louis, MO (US); Patrick D. McLAUGHLIN, St. Louis, MO (US); Juan Pedro Humberto ARHANCET, St. Louis, MO (US)

(73) Assignee: APSE, INC., St. Louis, MO (US)

(21) Appl. No.: 16/072,159

(22) PCT Filed: Jan. 31, 2017
(86) PCT No.: PCT/US2017/015861

§ 371 Date: Jul. 23, 2018

Related U.S. Application Data
(60) Provisional application No. 62/290,318, filed on Feb. 2, 2016.
(60) Provisional application No. 62/397,790, filed on Sep. 21, 2016.

(51) Int. Cl. A01N 63/02; C12N 15/70; C12N 15/81

(52) U.S. Cl. CPC A01N 63/02; C12N 2750/00023; C12N 15/81; C12N 15/70

(57) ABSTRACT

The invention describes recombinant sequences and methods for producing RNAs suitable for controlling proliferation of ant species, including Solenopsis, Camponotus, Linepithema, Tapinoma, Tetramorium, and Monomorium spp.

### CROSS REFERENCE TO RELATED APPLICATIONS

This application claims the benefit of U.S. Provisional Application No. 62/290,318 filed Feb. 2, 2016 and U.S. Provisional Application No. 62/397,790 filed Sep. 21, 2016.

### INCORPORATION OF SEQUENCE LISTING

A Sequence Listing is provided herewith as a text file entitled “Methods and Compositions for Controlling Ants_ST25.txt” created on Jan. 31, 2017 and having a size of 52 KB. The contents of the text file are incorporated by reference herein in their entirety.

### FIELD OF THE INVENTION

The invention comprises methods and compositions for RNA-mediated gene suppression as a way of controlling ants generally, and Solenopsis invicta (fire ants), Camponotus pennsylvanicus and Camponotus floridanus (carpenter ants), Linepithema humile (Argentine ants), Tapinoma sessile (odorous ants), Tetramorium caespitum (pavement ants), and Monomorium pharaonis (pharaoh ants), particularly. The methods and compositions have application in controlling proliferation of such ants without concomitant effect on related insects, plants or animal species.

### BACKGROUND OF THE INVENTION

RNA-mediated gene suppression (RNAi), first described in the nematode C. elegans, has been shown to be an effective method for modulating gene expression in many other organisms (Fire, et al., Potent and specific genetic interference by double-stranded RNA in Caenorhabditis elegans. Nature 391:806 (1998)). Feasibility of RNAi in controlling proliferation of insects affecting crops has been demonstrated using double-stranded RNA (dsRNA) by a number of research groups (reviewed in, Ivashuta, et al., Environmental RNAi in herbivorous insects. RNA 21:840 (2015)). Recombinant RNA constructs used for RNAi purposes described in the prior art generally consist of dsRNAs of about 18 to about 25 base pairs, but also include longer dsRNAs usually between about 100 to about 1,000 base pairs (bp). To successfully introduce dsRNA into insects, dsRNAs longer than or equal to approximately 60 bp are required for efficient uptake when supplied in the insect's diet (Bolognesi, et al., Ultrastructural Changes Caused by Snf7 RNAi in Larval Enterocytes of Western Corn Rootworm (Diabrotica virgifera virgifera Le Conte) PLoS One 7:e47534 (2012)). Long dsRNA molecules are cleaved in vivo into a diverse population of siRNAs by the host Dicer enzyme complex. Alternatively, RNAi gene suppression can also occur through the action of anti-sense RNAs directed to specific sequences via related processes. Practical application of RNAi methods for controlling insects in the field is limited by the cost of in vitro RNA synthesis and the chemical fragility of RNA to environmental and enzymatic degradation.

Bacteriophage MS2 capsid mediated delivery of toxins and imaging agents has been shown to be an effective method for delivering such agents to eukaryotic cells in vitro (Ashley, et al., Cell-specific delivery of diverse cargos by bacteriophage MS2 virus-like particles. ACS nano 5:5729 (2011)). Whether such bacteriophage capsids can serve a similar function for delivery of RNAi precursors to insects in the field is unknown. Effective delivery of RNAi precursors into target insects requires preventing non-specific RNA degradation, a facile route of administration, and the ability to release the RNAi precursors at the appropriate point within the target insect such that the RNAi precursors can be taken up by the insect cells and properly processed. Ideally, the RNAi precursor and delivery system must be economical and relatively simple to produce and distribute. The invention described here satisfies all these criteria with the added benefit of allowing rapid discovery, prototyping and commercial-scale production of new RNAi molecules.

Solenopsis spp. are known to be susceptible to RNAi introduced either by direct injection (Lu, et al., Oocyte membrane localization of vitellogenin receptor coincides with queen flying age, and receptor silencing by RNAi disrupts egg formation in fire ant virgin queens. FEBS Journal 276 3110 (2009)) or by feeding (M.-Y. Choi et al., Phenotypic impacts of PBAN RNA interference in an ant, Solenopsis invicta, and a moth, Helicoverpa zea. Journal of Insect Physiology 58:1159 (2012) on bait materials treated with RNAi precursors. However, field application of naked RNAs is generally impractical due to the sensitivity of RNA to environmental specific and non-specific degradation. Furthermore, RNA is highly susceptible to degradation during the course of feeding and transiting the insect gut. Both problems are especially true of single stranded RNAi precursors such as anti-sense RNA. The highly stable form of VLPs serves to protect RNA borne within the VLPs in vitro but the ability of a VLP to serve as a delivery system for anti-sense RNA and RNAi precursors is unknown. Additional questions include, is RNAi effective for inhibiting growth or colony formation of ant species other than Solenopsis spp.? Can targeted RNAi compositions discriminate between ant species? Is it possible to combine RNAi or anti-sense RNA compositions such that one RNAi composition might target Solenopsis spp. while sparing other ant species, while another RNAi composition might target Camponotus spp. while sparing other ant species? Are VLPs capable of effectively delivering RNAi precursors to the RNAi processing pathways, such as Dicer, of ants generally and Solenopsis and Camponotus spp. specifically? Can VLPs protect anti-sense RNA within the insect digestive tract and still deliver the intact anti-sense RNA into cells of the target insect? Can naked RNAi precursors be effectively formulated to allow direct ingestion by the target ant species?

### SUMMARY OF THE INVENTION

Methods of producing, packaging and/or purifying RNA within bacteriophage capsids (VLPs) are described in published U.S. Patent Application Nos. 2013/0167267, 2014/0302593 and U.S. Pat. No. 9,181,531, the contents of each incorporated herein by reference. The invention described here uses the unique properties of VLPs, (alternatively known as APSE RNA Containers, or “ARCs”), to provide an improved system for producing and delivering RNAi precursors to suppress expression of a target gene, preferably in an insect host, more preferably in ants. The preferred form of dsRNA for RNAi is a hairpin siRNA produced as a single transcribed RNA comprising an inverted repeat of the targeted host sequence separated by a non-homologous loop sequence such that a dsRNA forms is produced by annealing the homologous repeat sequences to allow proper processing of the dsRNA region by host DICER and DICER-like enzymes. Target organisms of particular interest are Solenopsis invicta commonly known as the red fire ant and the Camponotus species pennsylvanicus and floridanus commonly known as carpenter ants, Linepithema humile commonly known as Argentine ants, Monomorium pharaonis commonly known as pharaoh ants and household ants such as Tapinoma sessile commonly known as odorous ants and Tetramorium caespitum commonly known as pavement ants. Target genes of interest encode essential physiologic functions required for survival of an individual ant or of an ant colony in aggregate. RNAi methods of controlling fire ants, carpenter ants, Argentine ants, pharao ants, as well as various household ants are especially desired, since these ants represent major sources of economic and ecological damage.

An important advantage of producing RNAi precursors by the methods described here is that costly and complicated in vitro synthesis of RNA precursors is avoided and the desired RNA constructs can be produced by simple and economic fermentation methods. Production and purification of large quantities of RNAi precursors is facilitated by optionally coupling synthesis of the desired polynucleotide with expression of self-assembling bacteriophage capsid proteins, such as those of bacteriophage Qβ or MS2 to produce easily purified and relatively stable ARCs (VLPs) containing the desired polynucleotide, which may be applied directly to plant surfaces upon which the targeted insect pests feed, for example by spraying. Alternatively, encapsidated single-stranded RNAs can be produced stably and in large quantity and blended with other encapsidated single-stranded RNA to produce a desired molar ratios of each strand and the individual RNA strands isolated by removing the viral capsid coat protein and annealing the single-strands of RNA into the desired double-strand RNA product. In addition, encapsidated single-stranded RNAs may serve as a source of anti-sense RNA.

Once ingested, the ARCs may be digested in the course of transiting the insect host gut and the RNA molecules absorbed by cells lining the gut. In some cases, unencapsidated double-strand RNA may also transit the insect host gut without degradation and be absorbed by cells lining the gut. However, it is unlikely all species of RNA, especially single-stranded RNA will be able to avoid significant degradation unless packaged in an ARC. Within the target insect cells the RNAi precursors are processed by, among other things, the host Dicer enzyme complex to generate effective RNAi forms targeted against host gene transcripts to suppress expression of essential host genes. Examples of such essential genes include, without limitation, genes involved in controlling molting or other larval development events, actin or other cellular structural components, as well as virtually any gene related to replication, transcription or translation or other fundamental process required for viability. In addition, genes necessary to maintain colony integrity but not necessarily lethal to individual members of the colony may also be considered as essential and represent attractive targets for long term control of ant populations.

### DETAILED DESCRIPTION OF THE INVENTION

The present invention comprises DNA sequences, which when transcribed produce an RNAi precursor and mRNA translated into bacteriophage coat protein, which together assemble into uniquely stable VLPs or may be processed to produce isolated RNA. The VLPs and/or RNA may be purified in a form suitable for ingestion by feeding insects. Once ingested by target insects, the VLPs and RNAs transit the gut and are then assimilated into the insect cells where the RNAi precursor is processed into a form of RNAi that suppresses expression of a target gene important to insect viability. In some embodiments, suppression of such target genes is designed to result in death of the target insect. In another embodiment suppression of target genes is designed to produce sterile off-spring. In other embodiments suppression of the target gene results in colony collapse. Each of these embodiments may be used singly or in combination to produce the desired result. A key feature of the VLPs is that they are stable enough to protect the encapsidated RNAi precursors from degradation by non-specific environmental agents or by insect target cell RNAse enzymes, yet remain capable of introducing the RNAi precursors into the RNAi pathways in target insect cells after they are ingested.

The example DNA sequences presented here are designed to be ligated into suitable bacterial plasmid vectors as AsiSI-NotI restriction fragments. Such DNA sequences can be produced by direct synthesis or by sub-cloning the constituent fragments using techniques well known to those skilled in the art. The sequences may be modified as desired to manipulate specific restriction enzyme sites and incorporate alternative bacteriophage pac sequences. The specificity of the encoded anti-sense RNA and RNAi sequences may be modified to target different genes and insect hosts. Bacterial plasmid vectors containing transcriptional promoters capable of inducibly transcribing these DNA sequences include without limitation, bacteriophage T7 gene 1 promoter, bacteriophage T5 promoter and the bacteriophage lambda P_{L} and P_{R} promoters. Bacterial plasmid vectors such as pBR322, pUC derivatives and pACYC derivatives may also contain the bacteriophage Qβ or bacteriophage MS2 capsid protein coding sequence expressed from an inducible promoter. For expression in biological systems other than bacteria such as E. coli, equivalent vectors are known to those of ordinary skill in the art. Alternatively, such inducibly expressed capsid genes may be present on a separate bacterial plasmid compatible with the bacterial plasmid carrying the inducible RNAi precursor or anti-sense RNA encoding sequences. Such RNAi or anti-sense RNA molecules may be referred to as “RNA cargo molecules”. In an embodiment the inducibly expressed capsid gene may be integrated into the bacterial chromosome.

The production and purification of VLPs containing RNA cargo molecules are described in detail in U.S. Patent Application Nos. 2013/0167267, 2014/0302593 and in U.S. Pat. No. 9,181,531. Such methods are also described in U.S. Patent Application Nos. 2010/0167981, 2012/0046340, PCT/US12/71419, and PCT/US14/41111 and U.S. Pat. Nos. 5,443,969, and 6,214,982, the contents of each incorporated herein by reference. The VLPs produced by these methods can be processed a number of different ways known to those of ordinary skill in the art to facilitate application of such material for use in the field. In one embodiment the purified ARCs are further processed for spraying operations. In another embodiment the purified ARCs or the RNAs isolated therefrom are processed into a bait substance. Such processing may include spray drying, introduction of stabilizing or wetting agents, or forming an admixture of VLPs with other desired agents prior to application. Field applications may involve ground or arial spray methods, spot application, or incorporation and into bait materials.

A person of ordinary skill in the art understands that the invention may be targeted to different genes in different insect hosts by modifying the sequences described in the Examples below to reflect sequences of other genes in other target organisms. Thus, the invention provides a tool for determining the best RNAi target for suppressing a particular gene in any given target organism and a means for producing large quantities of RNAi targeting those genes. Further, the invention provides for methods of empirically determining which gene or group of genes may constitute the most effective RNAi target within a single insect or group of insects by screening the effectiveness of VLPs or RNAs containing various RNAi precursors targeted to specific genes or gene combinations by combinatory cloning methods. The invention also supports methods combining VLPs effective for control of certain insects in the field with different VLPs effective for control of other insects that may be present to tailor the insect control properties to those relevant at the point of application. The different insects may be of a different order, genus or species as those targeted by the original VLPs or RNAs, or may comprise RNAi resistant, or combinations of RNAi resistant populations, wherein the combination of one or more VLPs or RNAs targeting different genes within the target insect population ensures that no combination of RNAi resistance is likely to occur.

In one embodiment of the present invention, a first DNA sequence within a bacterial host is transcribed to produce a first RNA molecule encoding a bacteriophage coat protein. A second DNA sequence within the bacterial host is transcribed to produce a second RNA molecule comprising a bacteriophage pac site, followed by a sense sequence of a target gene from an insect, followed by a non-homologous RNA sequence capable of forming a single-stranded loop, followed by an anti-sense sequence complementary to the sense sequence of the target gene sequence, optionally followed by a second bacteriophage pac site. A single bacteriophage pac site may be present at the 5′ side of the second RNA molecule or may be present at the 3′ side of the second RNA molecule or may be situated within the non-homologous loop sequences of the second RNA molecule. The first RNA molecule is an mRNA which is translated by the bacterial host to produce a plurality of bacteriophage coat protein which, when physically associated with the second RNA molecule comprising the bacteriophage pac sequence(s) spontaneously forms VLPs, wherein the second RNA molecule is packaged within the VLP. The VLPs are isolated and further purified as necessary prior to application in the field. Alternatively, naked RNA is isolated from the VLPs or directly from the bacterial host and processed for field application. Target insects ingesting the naked RNA or the VLPs containing an RNAi precursor introduce the RNA molecule, whether borne within the VLP or not, into the host insect cells lining their gut. Once in the host insect cells the RNA may either be processed by the host insect cell's endogenous RNAi pathways or may function directly as an anti-sense RNA, resulting in anti-sense RNA- or RNAi-mediated suppression of gene expression of the host insect target gene. In one embodiment the insect is an ant. In other embodiments the ant is a Solenopsis spp. In a preferred embodiment the Solenopsis spp. is Solenopsis invicta. In other embodiments the ant is a Camponotus spp. In a preferred embodiment the Camponotus spp. is Camponotus pennsylvanicus. In another preferred embodiment the Camponotus spp. is Camponotus floridanus. In other embodiments the targeted ants are Argentine ants, pharao ants, odiferous ants or pavement ants. In some embodiments the RNAi may be targeted at all such ant species or to any particular combination of such ant species.

In an embodiment, a series of host bacteria containing a first DNA sequence encoding a bacteriophage coat protein and different second DNA sequences encoding various RNAi precursor sequences are isolated. Each isolated host bacteria is clonally expanded and the bacterial cell line archived. A sample of each bacterial cell line is subsequently outgrown and induced to transcribe the first and second DNA sequences, the VLPS are allowed to assemble within the host bacteria and the VLPs isolated therefrom. The RNA sequences within the series of resulting VLPs each encode a different antisense and optionally a complementary sense sequence homologous to different insect target genes or on different regions of a given insect target gene or on target genes from different insect targets altogether. Each of the different VLPs produced by the series of host bacteria is fed to target insects and their ability to suppress host insect gene expression measured, for example by scoring target insect mortality. Those VLPs producing the greatest level of RNAi-mediated suppression of gene expression represent the most effective RNA target for that particular target insect or position within a given target insect gene. Recourse to the corresponding bacterial cell line that produced each VLP allows rapid identification of the corresponding target sequence or gene. Likewise, recourse to the corresponding host bacterial cell line facilitates rapid scale-up of the desired VLP for RNAi-mediated suppression of gene expression of the host insect target gene for field application or further experimental investigation. One of ordinary skill in the art recognizes that random or pseudo-random collections of complementary DNA sequences based on insect genomic sequence data or for subsets of such genomic sequence encoding likely essential genes can be screened using multiplex or automated cloning technologies. A similar process can be employed to analyze target specificity of each RNAi candidate to ensure that non-targeted ant (or other insect species) species are not harmed by the RNAi composition.

In an embodiment the RNAi or anti-sense RNA suppresses expression of the gene encoding the ant vitellogenin receptor protein (VgR). In a preferred embodiment the RNAi or anti-sense RNA suppresses expression of the gene encoding VgR of Solenopsis invicta, but does not suppress expression of the gene encoding VgR in other ant species. In another preferred embodiment the RNAi or anti-sense RNA suppresses expression of the gene encoding VgR of Camponotus pennsylvanicus or C. floridanus, but does not suppress expression of the gene encoding VgR in other ant species. In other embodiments expression of the VgR gene of Argentine ants, pharao ants, odiferous ants or pavement ants is targeted.

In one embodiment the RNAi or anti-sense RNA suppresses expression of the gene encoding the ant telomerase variant X1 protein (TVX1). In a preferred embodiment the RNAi or anti-sense RNA suppresses expression of the gene encoding TVX1 of Solenopsis invicta, but does not suppress expression of the gene encoding TVX1 in other ant species. In another preferred embodiment the RNAi or anti-sense RNA suppresses expression of the gene encoding TVX1 of Camponotus pennsylvanicus or C. floridanus, but does not suppress expression of the gene encoding TVX1 in other ant species. In other embodiments expression of the TVX1 gene of Argentine ants, pharao ants, odiferous ants or pavement ants is targeted.

In an embodiment the RNAi or anti-sense RNA suppresses expression of the gene encoding the ant pheromone biosynthesis activating neuropeptide (PBAN). In a preferred embodiment the RNAi or anti-sense RNA suppresses expression of the gene encoding PBAN of Solenopsis invicta, but does not suppress expression of the gene encoding PBAN in other ant species. In another preferred embodiment the RNAi or anti-sense RNA suppresses expression of the gene encoding PBAN of Camponotus pennsylvanicus or C. floridanus, but does not suppress expression of the gene encoding PBAN in other ant species. In other embodiments expression of the PBAN gene of Argentine ants, pharao ants, odiferous ants or pavement ants is targeted.

In an embodiment the RNAi or anti-sense RNA suppresses expression of the gene encoding the ant pheromone biosynthetic activating neuropeptide receptor (PBANR). In a preferred embodiment the RNAi or anti-sense RNA suppresses expression of the gene encoding PBANR of Solenopsis invicta, but does not suppress expression of the gene encoding PBANR in other ant species. In another preferred embodiment the RNAi or anti-sense RNA suppresses expression of the gene encoding PBANR of Camponotus pennsylvanicus or C. floridanus, but does not suppress expression of the gene encoding PBANR in other ant species. In other embodiments expression of the PBANR gene of Argentine ants, pharao ants, odiferous ants or pavement ants is targeted.

In an embodiment the RNAi or anti-sense RNA suppresses expression of the gene encoding the ant wntless protein (WLS). In a preferred embodiment the RNAi or anti-sense RNA suppresses expression of the gene encoding WLS of Solenopsis invicta, but does not suppress expression of the gene encoding WLS in other ant species. In another preferred embodiment the RNAi or anti-sense RNA suppresses expression of the gene encoding WLS of Camponotus pennsylvanicus or C. floridanus, but does not suppress expression of the gene encoding WLS in other ant species. In other embodiments expression of the WLS gene of Argentine ants, pharao ants, odiferous ants or pavement ants is targeted.

In one embodiment the RNAi or anti-sense RNA suppresses expression of the gene encoding the ant multiple epidermal growth factor-like domains protein 10 (MEGF10). In a preferred embodiment the RNAi or anti-sense RNA suppresses expression of the gene encoding MEGF10 of Solenopsis invicta, but does not suppress expression of the gene encoding MEGF10 in other ant species. In another preferred embodiment the RNAi or anti-sense RNA suppresses expression of the gene encoding MEGF10 of Camponotus pennsylvanicus or C. floridanus, but does not suppress expression of the gene encoding MEGF10 in other ant species. In other embodiments the expression of the MEGF10 gene of Argentine ants, pharao ants, odiferous ants or pavement ants is targeted.

In an embodiment the RNAi or anti-sense RNA suppresses expression of the gene encoding the ant clatherin heavy chain protein (CHCP). In a preferred embodiment the RNAi or anti-sense RNA suppresses expression of the gene encoding CHCP of Solenopsis invicta, but does not suppress expression of the gene encoding CHCP in other ant species. In another preferred embodiment the RNAi or anti-sense RNA suppresses expression of the gene encoding CHCP of Camponotus pennsylvanicus or C. floridanus, but does not suppress expression of the gene encoding CHCP in other ant species. In other embodiments expression of the CHCP gene of Argentine ants, pharao ants, odiferous ants or pavement antsis targeted.

In an embodiment the RNAi or anti-sense RNA suppresses expression of the gene encoding the ant cell division cycle 7-related protein (CDC7). In a preferred embodiment the RNAi or anti-sense RNA suppresses expression of the gene encoding CDC7 of Solenopsis invicta, but does not suppress expression of the gene encoding CDC7 in other ant species. In another preferred embodiment the RNAi or anti-sense RNA suppresses expression of the gene encoding CDC7 of Camponotus pennsylvanicus or C. floridanus, but does not suppress expression of the gene encoding CDC7 in other ant species. In other embodiments expression of the CDC7 gene of Argentine ants, pharao ants, odiferous ants or pavement ants is targeted.

In an embodiment the RNAi or anti-sense RNA suppresses expression of the gene encoding the ant centrosomal protein 89 kdal (Cep89). In a preferred embodiment the RNAi or anti-sense RNA suppresses expression of the gene encoding Cep89 of Solenopsis invicta, but does not suppress expression of the gene encoding Cep89 in other ant species. In another preferred embodiment the RNAi or anti-sense RNA suppresses expression of the gene encoding Cep89 of Camponotus pennsylvanicus or C. floridanus, but does not suppress expression of the gene encoding Cep89 in other ant species. In other embodiments expression of the Cep89 gene of Argentine ants, pharao ants, odiferous ants or pavement ants is targeted.

In an embodiment the RNAi or anti-sense RNA suppresses expression of the gene encoding the ant beta subunit of the type-1 proteosome (PSMB1). In a preferred embodiment the RNAi or anti-sense RNA suppresses expression of the gene encoding PSMB1 of Solenopsis invicta, but does not suppress expression of the gene encoding PSMB1 in other ant species. In another preferred embodiment the RNAi or anti-sense RNA suppresses expression of the gene encoding PSMB1 of Camponotus pennsylvanicus or C. floridanus, but does not suppress expression of the gene encoding PSMB1 in other ant species. In other embodiments expression of the PSMB1 gene of Argentine ants, pharao ants, odiferous ants or pavement ants is targeted.

In an embodiment the RNAi or anti-sense RNA suppresses expression of the gene encoding the ant anamorsin protein. In a preferred embodiment the RNAi or anti-sense RNA suppresses expression of the gene encoding anamorsin of Solenopsis invicta, but does not suppress expression of the gene encoding anamorsin in other ant species. In another preferred embodiment the RNAi or anti-sense RNA suppresses expression of the gene encoding anamorsin of Camponotus pennsylvanicus or C. floridanus, but does not suppress expression of the gene encoding anamorsin in other ant species. In other embodiments expression of the the anamorsin protein gene of Argentine ants, pharao ants, odiferous ants or pavement ants is targeted.

In an embodiment the RNAi or anti-sense RNA suppresses expression of the gene encoding the ant actin 5C protein (A5C). In a preferred embodiment the RNAi or anti-sense RNA suppresses expression of the gene encoding A5C of Solenopsis invicta, but does not suppress expression of the gene encoding A5C in other ant species. In another preferred embodiment the RNAi or anti-sense RNA suppresses expression of the gene encoding A5C of Camponotus pennsylvanicus or C. floridanus, but does not suppress expression of the gene encoding A5C in other ant species. In other embodiments expression of the A5C gene of Argentine ants, pharao ants, odiferous ants or pavement ants is targeted.

In an embodiment the RNAi or anti-sense RNA suppresses expression of the gene encoding the ant beta actin protein. In a preferred embodiment the RNAi or anti-sense RNA suppresses expression of the gene encoding beta actin of Solenopsis invicta, but does not suppress expression of the gene encoding beta actin in other ant species. In another preferred embodiment the RNAi or anti-sense RNA suppresses expression of the gene encoding beta actin of Camponotus pennsylvanicus or C. floridanus, but does not suppress expression of the gene encoding beta actin in other ant species. In other embodiments expression of the beta actin gene of Argentine ants, pharao ants, odiferous ants or pavement ants is targeted.

In an embodiment the RNAi or anti-sense RNA suppresses expression of the gene encoding the ant ATP synthase delta subunit (ATPSD). In a preferred embodiment the RNAi or anti-sense RNA suppresses expression of the gene encoding ATPSD of Solenopsis invicta, but does not suppress expression of the gene encoding ATPSD in other ant species. In another preferred embodiment the RNAi or anti-sense RNA suppresses expression of the gene encoding ATPSD of Camponotus pennsylvanicus or C. floridanus, but does not suppress expression of the gene encoding ATPSD in other ant species. In other embodiments expression of the ATPSD gene of Argentine ants, pharao ants, odiferous ants or pavement ants is targeted.

In an embodiment the RNAi or anti-sense RNA suppresses expression of the gene encoding the ant Csp9 protein. In a preferred embodiment the RNAi or anti-sense RNA suppresses expression of the gene encoding Csp9 of Solenopsis invicta, but does not suppress expression of the gene encoding Csp9 in other ant species. In another preferred embodiment the RNAi or anti-sense RNA suppresses expression of the gene encoding Csp9 of Camponotus pennsylvanicus or C. floridanus, but does not suppress expression of the gene encoding Csp9 in other ant species. In other embodiments expression of the Csp9 gene of Argentine ants, pharao ants, odiferous ants or pavement ants is targeted.

In a further embodiment of the present invention the RNAi or anti-sense RNA suppression of gene expression is directed to targets for additional ant species to provide a broader spectrum of ant control. An important consideration for designing such broad spectrum ant control compositions is to minimize any chance of cross-reactivity with already vulnerable wasp and bee species. To identify targets for RNAi or anti-sense ant suppression with minimal potential for cross-reactivity with honeybees, conserved genes present in the selected ant genomes encoding likely essential genes were identified and aligned. Sequences unique among a series of ant species including, in addition to Solenopsis invicta (red fire ants) and Camponotus pennsylvanicus (Carpenter ants), an additional Carpenter ant species (Camponotus floridanus), Argentine ants (Linepithema humile), Odorous house ants (Tapinoma sessile), and Pharaoh ants (Monomorium pharaonsis), were screened against available honeybee and wasp genomes and essential genes with the least degree of homology between the ants and the wasps and bees identified. Of the available essential gene ant candidates two genes, encoding the clatherin heavy chain protein and ribosomal protein L32, were chosen as providing the best selectivity among the ant species and as having virtually no significant homology to wasp and bee homologs. In an embodiment, combinations of two or more of RNAi or anti-sense RNA directed to these genes from different ant species provides an effective method for controlling specific ant species without affecting non-target species, including other ant species.

The following Examples are illustrative of the invention and are not intended to limit the scope of the invention as described in detail above and as set out in the claims. Example 1 presents general tools and methods for practicing the invention. Example 2 presents a detailed description of use of such general tools and methods involving one targeted gene sequence (VgR) as representative of the invention. Subsequent Examples 3-16 present additional instances of the invention. In particular, knowledge of the targeted gene sequences presented in Examples 3-16 is sufficient to allow one of ordinary skill in the art to use the tools and methods described in Example 1 to generate compositions analogous to those presented in Example 2 to achieve the overall goals of general and/or selective control of various ant species.

### EXAMPLE 1

### Expression Constructs and General Assay Procedures

RNAi and anti-sense RNA for suppressing targeted gene expression are produced by an E. coli based expression system. In each case, the desired RNAi precursor is obtained as a synthetic DNA sequence comprising (5′-3′) an AsiSI restriction site sequence, the targeted gene sequence, a 150 ntd loop sequence, a sequence homologous to the targeted gene sequence, and a NotI restriction site sequence. The synthetic DNA sequence is digested with AsiSI and NotI and ligated into plasmid pAPSE10136 (SEQ ID NO. 1).

Plasmid pAPSE10136 is based on plasmid pBR322 and contains two T7 expression domains separated by T7 terminators. One of these expression domains comprises a T7 promoter sequence upstream of a single copy of the bacteriophage MS2 coat protein gene followed by a T7 terminator. The second expression domain comprises a T7 promoter sequence upstream of unique AsiSI and NotI restriction sites (which are bracketed by one pac site 5′ of the cloning sites and one pac site 3′ of the cloning site) followed by an MS2 packaging sequence which is followed by a T7 terminator. Ligation of any sequence into the AsiSI-NotI region and induction of the T7 promoters produces both MS2 capsid protein and an untranslated RNA transcript comprising the AsiSI-NotI sequences fused to an MS2 pac site sequence. Upon induction, the pac site sequence of the untranslated RNA transcript interacts with the MS2 capsid protein to induce formation of VLPs encapsidating the RNA transcript. Recovery and purification of the VLPs from the induced cultures, and optionally isolating the RNA from the purified VLPs, provides a source of the desired RNAi precursor or anti-sense RNA for subsequent gene suppression studies.

A simple bioassay of the ability of the purified VLPs or RNAs to suppress essential gene expression and thereby directly kill ants, involves feeding a defined number of worker ants the test substance at known concentration in 10% sucrose solution and measuring the mortality rate of the worker ants. Under test conditions mortality at twelve days for ants fed a 10% sucrose solution with no added test RNA is 25%. This assay and variants thereof may be referred to as the worker mortality assay.

A more complex assay, designed to measure the ability of the purified VLPs or RNAs to disrupt colony integrity to provide long term eradication comprise bioassays similar to those described by Lu, et al. Newly emerged virgin queens from laboratory colonies are kept in a 3-cm diameter plate nest with holes on the lid small enough to isolate the virgin queens but large enough to receive care from workers within the queenright colony and allow exposure to primer pheromone from nearby mated queens. Newly mated queens are collected from the field after mating flights at 3-4 p.m. and are housed in containers adjacent to the virgin queens to allow phermone priming, while avoiding direct physical interaction between the queens (and between the nurse workers and the mated and virgin queens).

Purified or encapsidated RNA is dissolved in 10% sucrose solution (wt./vol.) to a known concentration and provided for a period of 12 days via a capillary tube to nurse workers that are caring for queen larvae. Silencing of essential genes in virgin queens through RNA interference, either by feeding encapsidated or naked dsRNA targeting VgR, can abolish egg formation, result in deformities comprising the virgin queens ability to mate, or otherwise interfere with colony propagation. Variations of this “virgin queen bioassay” can be utilized for determining RNAi suppression of other genes.

### EXAMPLE 2

### Control of Ant Species by VLPs Containing RNAi Precursors for VgR Gene Suppression

The gene encoding the vitellogenin receptor protein (the VgR gene) can be targeted by RNAi to control ant populations based on divergence of the gene sequence in ant species relative to other insects. In addition, at least one region of the Solenopsis invicta VgR gene (NCBI Reference Sequence XM_011168460) is unique among ants to Solenopsis invicta including 691 nucleotides at positions 2055-2745 (SEQ ID NO. 2). This region was also isolated as two more or less equal sized RNAi precursors, the 345 nucleotides at positions -2055-2400 (SEQ ID NO. 3), and the 344 nucleotides at positions 2401-2745 (SEQ ID NO. 4). Use of RNAi compositions based on these sequences allows selective control of Solenopsis invicta without harm to ant species lacking significant homology to these sequences

A 1548 nucleotide synthetic DNA construct (SEQ ID NO. 5) encoding an inverted repeat comprising sense and anti-sense copies of the 691 bp unique region of the ant VgR gene with the repeat copies separated by a 150 base pair non-homologous loop sequence flanked by AsiSI and NotI restriction sites was designed and synthesized by Life Technologies' GeneArt gene synthesis service (Life Technologies, Grand Island, N.Y.). The construct was inserted as an AsiSI-NotI restriction fragment into the corresponding restriction sites of plasmid pAPSE10136 to form the corresponding expression plasmid pAPSE 10397 (SEQ ID NO. 6).

The expression plasmid (SEQ ID NO. 6) is transformed into E. coli host strain HTE115(DE3) (Timmons, et al., Ingestion of bacterially expressed dsRNAs can produce specific and potent genetic interference in Caenorhabditis elegans. Gene 263:103-12 (2001)). Ampicillin resistant clones of the transformed bacterial strain are selected on LB agar plates. The selected clones are subsequently grown at 37° C. in 100 ml of LB media containing ampicillin until the culture reached OD₆₀₀ 0.8, at which time isopropyl β-D-thiogalactopyranoside is added to a final concentration of 1 mM to induce T7 polymerase directed transcription of the MS2 capsid protein and the VgR RNA precursor. The induced cultures are allowed to grow for at least 4 hours post-induction to allow sufficient time for VLP formation. Cells are collected by centrifugation at 3,000 g at 4° C. Each pellet is stored at 4° C. until processing.

VLPs containing the 691 bp stem RNAi precursor targeted against the ant VgR gene are purified by re-suspending each pellet in approximately 10 volumes of 20 mM Tris-HCl, pH 7.0, containing 10 mM NaCl and sonicated to lyse the cells. Cell debris is removed by centrifugation at 16,000 g. Each sample is further processed by addition of Benzonase® Nuclease (Sigma Aldrich, St. Louis, Mo.) added to a final concentration of about 100 units per mL and incubated at 37° C. for two hours. Proteinase K is then added to final concentration of 150 micrograms per mL and incubated at 37° C. for an additional three hours. A saturated ammonium sulfate solution is prepared by adding ammonium sulfate to water to a final concentration of 4.1 M. The saturated ammonium sulfate is added to the enzymatically treated VLPs to a final concentration of 186 mM (approximately a 1:22 dilution) and placed on ice for two hours. Unwanted precipitate is cleared from the lysate by centrifugation at 16,000 g. A second precipitation is conducted by addition of 155 mg of dry ammonium sulfate directly to each mL of cleared lysate. Each sample is vortexed and incubated on ice for two hours. Each precipitate is spun down at 16,000 g and the solid precipitate resuspended in one tenth the original volume of 20 mM Tris-HCl, pH 7.0, containing 10 mM NaCl.

The other two RNAi precursor sequences targeted against the VgR gene were prepared as described above. An 858 ntd synthetic DNA sequence comprising 345 nucleotide sense and anti-sense sequences homologous to SEQ ID NO. 3 and separated by a 150 ntd non-homologous loop was synthesized (SEQ ID NO. 7) and inserted as an AsiSI-NotI restriction fragment into the corresponding restriction sites of plasmid pAPSE10136 to form the expression plasmid pAPSE10426 SEQ ID NO. 8 (SEQ ID NO. 8). Upon induction of the T7 promoter(s), this plasmid produces an RNA transcript comprising a 345 bp stem RNAi precursor. Another synthetic DNA sequence, 856 ntds long, comprising 344 nucleotide sense and anti-sense sequences homologous to SEQ ID NO. 4 and separated by a 150 ntd non-homologous loop was also synthesized (SEQ ID NO. 9) and inserted as a AsiSI-NotI restriction fragment into the corresponding restriction sites of plasmid pAPSE10136 to form the expression plasmid pAPSE10427 (SEQ ID NO. 10). Upon induction of the T7 promoter(s), this plasmid produces an RNA transcript comprising a 344 bp stem RNAi precursor. Expression plasmids SEQ ID NO. 8 and SEQ ID NO. 10 are separately transformed into E. coli host strain HTE115(DE3) and VLPs isolated from each of the two transformed strains, containing 345 and 344 bp stem RNAi precursors respectively, targeted against Solenopsis invicta VgR. In total, 3 different VLP variants are produced, comprising 691, 344, and 344 base pair RNAi precursors to suppress VgR expression.

Bioassays for reduced vitellogenin production are performed similar to those described by Lu, et al., and described in Example 1. Silencing of the VgR gene in virgin queens through RNAi, either by feeding encapsidated or naked dsRNA targeting VgR can abolish egg formation, thus directly demonstrating effective control of ants.

### EXAMPLE 3

### Control of Ant Species by VLPs Containing RNAi Precursors for Telomerase Gene Suppression

The gene encoding the telomerase protein can also be effectively targeted by RNAi to control ant populations in general based on divergence of the gene sequence in ant species relative to other insects. In addition, at least five segments of the Solenopsis invicta gene encoding telomerase (NCBI sequence identifier XM_011174575) are unique to Solenopsis invicta including the 226 nucleotides at positions 124-349 (SEQ ID NO. 11), the 105 nucleotides at positions 375-479 (SEQ ID NO. 12), the 147 nucleotides at positions 532-678 (SEQ ID NO. 13), the 207 nucleotides at positions 885-679 (SEQ ID NO. 14) and the 114 nucleotides at positions 1035-1148 (SEQ ID NO. 15). Synthetic DNA sequences comprising an inverted repeat of sense and anti-sense sequences homologous to the unique regions of the telomerase gene identified above, separated by a 150 base non-homologous loop sequence, are constructed and cloned into pAPSE10136. VLPs produced as described in Examples 1 and 2 are obtained. The ability of the resulting encapsidated RNAi precursors to inhibit ant reproduction is determined using the virgin queen bioassay. Failure to produce viable larvae indicates that telomerase represents a viable RNAi target for ant and ant colony eradication.

### EXAMPLE 4

### Control of Ant Species by VLPs Containing RNAi Precursors for Pheromone Biosynthesis Activating Neuropeptide Gene Suppression

The gene encoding the pheromone biosynthesis activating neuropeptide (PBAN) can also be effectively targeted by RNAi to control ants in general based on divergence of the gene sequence in ant species relative to other insects. In addition, at least one segment of the Solenopsis invicta gene encoding PBAN (NCBI sequence identifier NM_001304598.1) is unique to Solenopsis invicta including the 108 nucleotides at positions 116-223 (SEQ ID NO. 16). A synthetic DNA sequence comprising an inverted repeat of sense and anti-sense sequences homologous to the unique regions of the PBAN gene identified above, separated by a 150 base non-homologous loop sequence, is constructed and cloned into pAPSE10136. VLPs produced as described in Examples 1 and 2 are obtained. The ability of the resulting encapsidated RNAi precursors to increase mortality in ants is determined using the worker mortality bioassay. In addition to mortality, PBAN inhibition is predicted to aid in colony collapse by disrupting the ability of foraging ants to navigate (and thus locate the colony) by loss of their ability to construct a phermone trail.

### EXAMPLE 5

### Control of Ant Species by VLPs Containing RNAi Precursors for Pheromone Biosynthesis Activating Neuropeptide Receptor Gene Suppression

The gene encoding the pheromone biosynthesis activating neuropeptide receptor (PBANR) can also be effectively targeted by RNAi to control ants in general based on divergence of the gene sequence in ant species relative to other insects. In addition, at least four segments of the Solenopsis invicta gene encoding PBANR (NCBI sequence identifier JX657040) are unique to Solenopsis invicta including the 224 nucleotides at positions 24-247 (SEQ ID NO. 17), the 141 nucleotides at positions 551-691 (SEQ ID NO. 18), the 109 nucleotides at positions 1462-1570 (SEQ ID NO. 19), and the 106 nucleotides at positions 1613-1718 (Figure T; SEQ ID NO. 20). Synthetic DNA sequences, each comprising an inverted repeat of sense and anti-sense sequences homologous to the unique regions of the PBAN gene identified above, separated by a 150 base non-homologous loop sequence, are constructed and cloned into pAPSE10136. VLPs produced as described in Examples 1 and 2 are obtained. The ability of the resulting encapsidated RNAi precursors to increase mortality in ants is determined using the worker mortality bioassay. In addition to individual mortality, PBAN inhibition is predicted to aid in colony collapse by disrupting the ability of foraging ants to navigate (and thus relocate the colony) by loss of their ability to construct a phermone trail.

### EXAMPLE 6

### Control of Ant Species by VLPs Containing RNAi Precursors for Wntless Gene Suppression

The gene encoding the wntless protein can also be effectively targeted by RNAi to control ants in general based on divergence of the gene sequence in ant species relative to other insects. In addition, at least two segments of the Solenopsis invicta gene encoding the wntless protein (NCBI sequence identifier XM_011162771.1) are unique to Solenopsis invicta including the 141 nucleotides at positions 292-431 (SEQ ID NO. 21) and the 177 nucleotides at positions 1489-1665 (SEQ ID NO. 22). Synthetic DNA sequences, each comprising an inverted repeat of sense and anti-sense sequences homologous to the unique regions of the gene encoding wntless identified above, separated by a 150 base non-homologous loop sequence, are constructed and cloned into pAPSE10136. VLPs produced as described in Examples 1 and 2 are obtained. The ability of the resulting encapsidated RNAi precursors to inhibit ant reproduction may be determined using a variation of the virgin queen bioassay as described in Example 2. Failure to produce winged ants indicates that wntless represents a viable RNAi target for ant colony eradication. Failure to develop wings precludes the ability to engage in mating flights which may lead to colony collapse and will limit the ability of ants within the ecolony to disperse and establish new colonies.

The ability of the resulting encapsidated RNAi precursors to inhibit ant reproduction is determined using the virgin queen bioassay as described in Example 2. Failure to produce viable larvae indicates that the wntless gene represents a viable RNAi target for ant colony eradication.

### EXAMPLE 7

### Control of Ant Species by VLPs Containing RNAi Precursors for Multiple Epidermal Growth Factor-Like Domains Protein 10 Gene Suppression

The gene encoding the multiple epidermal growth factor-like domains protein 10 (MEGF10) can also be effectively targeted by RNAi to control ant populations in general based on divergence of the gene sequence in ant species relative to other insects. In addition, at least one segment of the Solenopsis invicta gene encoding MEGF10 (NCBI sequence identifier XM_011169520.1) is unique to Solenopsis invicta including the 137 nucleotides at positions 1712-1848 (SEQ ID NO. 23). A synthetic DNA sequence comprising an inverted repeat of sense and anti-sense sequences homologous to the unique region of the MEGF10 gene identified above, separated by a 150 base non-homologous loop sequence, is constructed and cloned into pAPSE10136. VLPs produced as described in Examples 1 and 2 are obtained. The ability of the resulting encapsidated RNAi precursors to disrupt ant colonies is determined using the worker mortality bioassay. Suppression of MEGF10 is predicted to produce severe neurological effects leading to death of individual ants as well as colony disruption. In addition, suppression of MEGF10 disrupts normal larvae development.

### EXAMPLE 8

### Control of Ant Species by VLPs Containing RNAi Precursors for Clatherin Heavy Chain Protein Gene Suppression

The gene encoding the clatherin heavy chain protein (NCBI sequence identifier XM_0111613001) can be effectively targeted by RNAi to control ant populations in general based on divergence of the gene sequence in ant species relative to the gene sequences found in other Hymanopteran insects. The sequences at nucleotides 3212-3314 (SEQ ID NO. 24) represent an RNAi target unique among ants to Solenopsis invicta as do sequences at nucleotides 2022-2092 (SEQ ID NO. 25) and at nucleotides 4794-4894 (SEQ ID NO. 26). A synthetic DNA sequence comprising an inverted repeat of sense and anti-sense sequences homologous to any one of the unique regions of the clatherin heavy chain protein gene identified above, separated by a 150 base non-homologous loop sequence, is constructed and cloned into pAPSE10136. VLPs produced as described in Examples 1 and 2 are obtained. The ability of the resulting encapsidated RNAi precursors to control ants is determined using the worker mortality bioassay. VLPs comprising SEQ ID NO. 24 (provided at a concentration of 500 mg/L in 10% sucrose solution) produced a twelve day mortality rate of 84%.

The gene encoding the clatherin heavy chain protein for other ant species such as Camponotus floridanus (Carpenter ants), Linepithema humile (Argentine ants), Tapinoma sessile (Odorous house ants), and Monomorium pharaonsis (Pharaoh ants) also encodes sequences unique among the ants and entirely missing in wasp and bee gene homologs. These represent RNAi targets specific to each individual ant species. For example, the clatherin heavy chain protein gene of C. floridanus (NCBI sequence identifier XM_0112680471) sequences at nucleotides 2036-2106 (SEQ ID NO. 27) and 4808-4908 (SEQ ID NO. 28)can be synthesized as described above to produce VLPs as described to specifically control C. floridanus. The clatherin heavy chain protein gene sequence of L. humile (NCBI sequence identifier XM_0123694721) at nucleotide positions 1951-2021 (SEQ ID NO. 29) and 4723-4823 (SEQ ID NO. 30) are specific to Argentine ants and synthetic constructs as described herein containing these sequences are specific to control of Argentine ants. Likewise, the clatherin heavy chain protein gene sequence of M. pharaonsis (NCBI sequence identifier XM_0126717651 at nucleotide positions 2152-2222 (SEQ ID NO. 31) and 4924-4894 (SEQ ID NO. 32) are specific to Pharaoh ants and synthetic constructs as described herein containing these sequence are specific to control of Pharoah ants.

### EXAMPLE 9

### Control of Ant Species by VLPs Containing RNAi Precursors for Cell Division Cycle 7-Related Protein Kinase Gene Suppression

The gene encoding the cell division cycle 7-related protein kinase (CDC7) can also be effectively targeted by RNAi to control ant populations in general based on divergence of the gene sequence in ant species relative to other insects. In addition, at least three segments of the Solenopsis invicta gene encoding CDC7 (NCBI sequence identifier XM_011165089.1) are unique to Solenopsis invicta including the 127 nucleotides between positions 175-301 (SEQ ID NO. 33), the 101 nucleotides between positions 703-803 (SEQ ID NO. 34), and the 107 nucleotides between positions 796-902(SEQ ID NO. 35). Synthetic DNA sequences comprising an inverted repeat of sense and anti-sense sequences homologous to the unique region of the CDC7 gene identified above, separated by a 150 base non-homologous loop sequence, are constructed and cloned into pAPSE 10136. VLPs produced as described in Examples 1 and 2 are obtained. The ability of the resulting encapsidated RNAi precursors to control ants is determined using the worker mortality bioassay.

### EXAMPLE 10

### Control of Ant Species by VLPs Containing RNAi Precursors for Centrosomal Protein 89 kdal Gene Suppression

The gene encoding the centrosomal protein 89 kdal (Cep89) can also be effectively targeted by RNAi to control ant populations in general based on divergence of the gene sequence in ant species relative to other insects. At least two segments of the Solenopsis invicta gene (NCBI sequence identifier XM_011175256.1) encoding Cep89 are unique to Solenopsis invicta including the 119 nucleotides between positions 461-579 (SEQ ID NO. 36) and the 105 nucleotides between positions 1506-1610 (SEQ ID NO. 37). Synthetic DNA sequences comprising an inverted repeat of sense and anti-sense sequences homologous to the unique region of the Cep89 gene identified above, separated by a 150 base non-homologous loop sequence, is constructed and cloned into pAPSE 10136. VLPs produced as described in Examples 1 and 2 were obtained. The ability of the resulting encapsidated RNAi precursors to control ants was determined using the worker mortality bioassay. VLPs comprising SEQ ID NO. 28 and SEQ ID NO. 29 (provided at a concentration of 100 mg/L in 10% sucrose solution) produced a twelve day mortality rate of 83%.

### EXAMPLE 11

### Control of Ant Species by VLPs Containing RNAi Precursors for Type-1 Proteosome Beta-Subunit Protein Gene Suppression

The gene encoding the beta subunit of the type-1 proteosome can also be effectively targeted by RNAi to control ant populations in general based on divergence of the gene sequence in ant species relative to other insects. At least two segments of the Solenopsis invicta gene (NCBI sequence identifier XM_011159663.1) are unique to Solenopsis invicta including the 153 nucleotides between positions 74-226 (SEQ ID NO. 38) and the 137 nucleotides between positions 885-1021 (SEQ ID NO. 39). Synthetic DNA sequences, each comprising an inverted repeat of sense and anti-sense sequences homologous to the unique region of the gene encoding the beta subunit of type-1 proteosome identified above, separated by a 150 base non-homologous loop sequence, were constructed and cloned into pAPSE10136. VLPs produced as described in Examples 1 and 2 were obtained. The ability of the resulting encapsidated RNAi precursors to control ants was determined using the worker mortality bioassay. VLPs comprising SEQ ID NO. 38 and SEQ ID NO. 39 (provided at a concentration of 500 mg/L in 10% sucrose solution) produced a twelve day mortality rate of 69%.

### EXAMPLE 12

### Control of Ant Species by VLPs Containing RNAi Precursors for Anamorsin Gene Suppression

The gene encoding anamorsin (an apoptosis inhibitor) can also be effectively targeted by RNAi to control ant populations in general based on divergence of the gene sequence in ant species relative to other insects. At least two segments of the Solenopsis invicta (NCBI sequence identifier XM_011161006.1) gene are unique to Solenopsis invicta including the 220 nucleotides between positions 319-538 (SEQ ID NO. 40) and the 110 nucleotides between positions 996-1105 (SEQ ID NO. 41). Synthetic DNA sequences, each comprising an inverted repeat of sense and anti-sense sequences homologous to the unique regions of the anamorsin gene identified above, separated by a 150 base non-homologous loop sequence, is constructed and cloned into pAPSE 10136. VLPs produced as described in Examples 1 and 2 are obtained. The ability of the resulting encapsidated RNAi precursors to control ants is determined using the worker mortality bioassay.

### EXAMPLE 13

### Control of Ant Species by VLPs Containing RNAi Precursors for Actin 5C Gene Suppression

The actin 5C gene can also be effectively targeted by RNAi to control ant populations in general based on divergence of the gene sequence in ant species relative to other insects. At least two segments of the Solenopsis invicta gene (NCBI sequence identifier XM_011161006.1) are unique to Solenopsis invicta including the 300 nucleotides between positions -16-283 (SEQ ID NO. 42). Synthetic DNA sequences, each comprising an inverted repeat of sense and anti-sense sequences homologous to the unique regions of the actin 5C gene identified above, separated by a 150 base non-homologous loop sequence, are constructed and cloned into pAPSE 10136. VLPs produced as described in Examples 1 and 2 are obtained. The ability of the resulting encapsidated RNAi precursors to control ants is determined using the worker mortality bioassay.

### EXAMPLE 14

### Control of Ant Species by VLPs Containing RNAi Precursors for β-Actin Gene Suppression

The β-actin gene can also be effectively targeted by RNAi to control ant populations in general based on divergence of the gene sequence in ant species relative to other insects. At least one segment of the Solenopsis invicta gene (NCBI sequence identifier XM_011175337.1) is unique to Solenopsis invicta comprising the 300 nucleotides between positions -49-250 (SEQ ID NO. 43). A synthetic DNA sequence comprising an inverted repeat of sense and anti-sense sequences homologous to the unique region of the β-actin gene identified above, separated by a 150 base non-homologous loop sequence, was constructed and cloned into pAPSE10136. VLPs produced as described in Examples 1 and 2 were obtained. The ability of the resulting encapsidated RNAi precursors to control ants was determined using the worker mortality bioassay described in Example 2. The ability of the resulting encapsidated RNAi precursors to control ants was determined using the worker mortality bioassay. VLPs comprising SEQ ID NO. 35 (provided at a concentration of 100 mg/L in 10% sucrose solution) produced a twelve day mortality rate of 90%.

### EXAMPLE 15

### Control of Ant Species by VLPs Containing RNAi Precursors for Suppression of the Gene Encoding the Delta Subunit of ATP Synthase

The gene encoding the delta subunit of ATP synthase can also be effectively targeted by RNAi to control ant populations in general based on divergence of the gene sequence in ant species relative to other insects. At least one segment of the Solenopsis invicta gene (NCBI sequence identifier XM_011158204.1) is unique to Solenopsis invicta comprising the 387 nucleotides between positions 1-387 (SEQ ID NO. 44). A synthetic DNA sequence comprising an inverted repeat of sense and anti-sense sequences homologous to the unique region of the gene encoding the delta subunit of ATP synthase identified above, separated by a 150 base non-homologous loop sequence, was constructed and cloned into pAPSE10136. VLPs produced as described in Examples 1 and 2 were obtained. The ability of the resulting encapsidated RNAi precursors to control ants was determined using the worker mortality bioassay. VLPs comprising SEQ ID NO. 36 (provided at a concentration of 500 mg/L in 10% sucrose solution) produced a twelve day mortality rate of 81%.

### EXAMPLE 16

### Control of Ant Species by VLPs Containing RNAi Precursors for Csp9 Gene Suppression

The Csp9 gene, regulating integument and moulting, can also be effectively targeted by RNAi to control ant populations in general based on divergence of the gene sequence in ant species relative to other insects. At least one segment of the Solenopsis invicta gene (NCBI sequence identifier EE129471.1) is unique to Solenopsis invicta comprising the 238 nucleotides between positions 46-283 (SEQ ID NO. 45). A synthetic DNA sequence comprising an inverted repeat of sense and anti-sense sequences homologous to the unique region of the Csp9 gene identified above, separated by a 150 base non-homologous loop sequence, was constructed and cloned into pAPSE10136. VLPs produced as described in Examples 1 and 2 are obtained. The ability of the resulting encapsidated RNAi precursors to control ants is determined using the worker mortality bioassay.

### EXAMPLE 17

### Control of Ant Species by VLPs Containing RNAi Precursors for rpL32 Gene Suppression

The rpL32 gene, encoding a protein critical for the assembly and maturation of rRNA into a functional ribosome, can also be effectively targeted by RNAi to control ant populations in general based on divergence of the gene sequence among ant species and relative to other Hymanopteran insects. At least one segment of the Solenopsis invicta gene (NCBI sequence identifier EE129471.1) is unique to Solenopsis invicta comprising the 238 nucleotides between positions 46-283 (SEQ ID NO. 45). Two additional segments of the Solenopsis invicta gene (NCBI sequence identifier XM_0111755071) at nucleotides 464-536 (SEQ ID NO. 46) and 452-532 (SEQ ID NO. 47) represent unique RNAi or anti-sense RNA targets for controlling fire ants. A region of the rpL32 gene of C. floridanus (NCBI sequence identifier XM_0112570271) including nucleotide positions 417-489 (SEQ ID NO. 48) and 405-485 (SEQ ID NO. 49) represent unique RNAi or anti-sense RNA targets for controlling Carpenter ants. A region of the rpL32 gene of L. humile (NCBI sequence identifier XM_0123800581) including nucleotide positions 433-505 (SEQ ID NO. 50) and 421-501 (SEQ ID NO. 51) represent unique RNAi or anti-sense RNA targets for controlling Argentine ants. A region of the rpL32 gene of M pharaonsis (NCBI sequence identifier XM_0126715791) including nucleotide positions 459-531 (SEQ ID NO. 52) and 463-543 (SEQ ID NO. 53) represent unique RNAi or anti-sense RNA targets for controlling Pharaoh ants.

Synthetic DNA sequence comprising an inverted repeat of sense and anti-sense sequences homologous to the unique regions of the rpL32 gene identified above, separated by a 150 base non-homologous loop sequence, are constructed and cloned into pAPSE10136. VLPs produced as described in Examples 1 and 2 are obtained. The ability of the resulting encapsidated RNAi precursors to control ants is determined using the worker mortality bioassay.

1. A method for controlling a target insect comprising, transforming a microbial host with a first DNA sequence comprising a gene encoding a bacteriophage capsid protein and a second DNA sequence encoding an RNA transcript comprising at least one bacteriophage pac sequence coupled to an RNAi precursor sequence, inducing the microbial host to express the first and second DNA sequences, isolating RNAi precursor or virus-like particles (VLPs) comprising the capsid protein and RNAi precursor from the microbial host, and contacting the isolated RNAi precursor or VLPs with the target insect.

2. The first DNA sequence of claim 1, wherein the bacteriophage capsid protein derives from a levivirus.

3. The first DNA sequence of claim 2, wherein the levivirus is Qβ.

4. The first DNA sequence of claim 2, wherein the levivirus is MS2.

5. The second DNA sequence of claim 1, wherein the RNAi precursor sequence comprises sequence homologous to sequences selected from the group comprising SEQ ID NOs. 2-4 and 11-53.

6. The second DNA sequence of claim 1, wherein said DNA sequence encodes an RNAi precursor comprising a hairpin siRNA.

7. The second DNA sequence of claim 1, wherein said DNA encodes an RNAi precursor comprising an antisense RNA.

8. The microbial host of claim 1, wherein the first DNA sequence and second DNA sequence are on separate episomes.

9. The microbial host of claim 1, wherein the first DNA sequence and the second DNA sequence are on the same episome.

10. The microbial host of claim 1, wherein one of the first DNA sequence and the second DNA sequence is integrated into the bacterial host chromosome.

11. The microbial host of claim 1, wherein both the first DNA sequence and the second DNA sequence are integrated into the bacterial host chromosome.

12. The microbial host of claim 1, wherein the microbial host is a bacterium.

13. The bacterium of claim 11, wherein the bacterium is Escherichia coli.

14. The microbial host of claim 1, wherein the microbial host is a yeast.

15. The yeast of claim 13, wherein the yeast is Saccharomyces cerevisiae.

16. The method of controlling the target insect of claim 1, wherein the target insect is an ant.

17. The method of controlling the target insect of claim 1, wherein the target insect comprises Camponotus spp.

18. The method of controlling the target insect of claim 1, wherein the target insect comprises Camponotus pennsylvanicus.

19. The method of controlling the target insect of claim 1, wherein the target insect comprises Camponotus.floridanus.

20. The method of controlling the target insect of claim 1, wherein the target insect comprises Linepithema humile.

21. The method of controlling the target insect of claim 1, wherein the target insect comprises Tapinoma sessile.

22. The method of controlling the target insect of claim 1, wherein the target insect comprises Tetramorium caespitum.

23. The method of controlling the target insect of claim 1, wherein the target insect comprises Monomorium pharaonic.

24. The method of controlling the target insect of claim 1, wherein the target insect comprises Solenopsis spp.

25. The method of controlling the target insect of claim 1, wherein the target insect comprises Solenopsis invicta.

26. A VLP comprising a heterologous cargo molecule, wherein the heterologous cargo molecule comprises an RNAi precursor.

27. A VLP comprising a heterologous cargo molecule, wherein the heterologous cargo molecule comprises an anti-sense RNA.

28. A VLP comprising a heterologous cargo molecule, wherein the heterologous cargo molecule comprises an RNAi precursor and further comprises a sense strand sequence followed by a non-homologous loop sequence followed by an anti-sense sequence homologous to the sense strand sequence and a bacteriophage packaging sequence.

29. The VLP of claim 28, wherein the sense strand sequence of the RNAi precursor is selected from the group consisting of SEQ ID NOs. 2-4 and 11-53.

30. A VLP comprising a heterologous cargo molecule, wherein the heterologous cargo molecule comprises an anti-sense RNA and a bacteriophage packaging sequence.

31. The VLP of claim 30, wherein the sequence of the anti-sense RNA is homologous to sequences selected from the group consisting of SEQ ID NOs. 2-4 and 11-53.

32. A composition comprising an RNA, wherein the RNA is further comprised of sequences selected from the group consisting of SEQ ID NOs. 2-4 and 11-53 covalently joined to a bacteriophage packaging sequence.
